(19) United States
(12) Patent Application Publication
(10) Pub. No.: US 2022/0370607 A1
(43) Pub. Date: Nov. 24, 2022

(54) INHIBITOR, INHIBITOR COMPOSITION, DRUG AND USE THEREOF

(71) Applicant: SOOCHOW UNIVERSITY, Suzhou (CN)

(72) Inventors: Lin YANG, Suzhou (CN); Zixuan LI, Suzhou (CN)

(21) Appl. No.: 17/631,034

(22) PCT Filed: Jan. 29, 2021
(86) PCT No.: PCT/CN2021/074392

§ 371 Date: Jan. 28, 2022

(30) Foreign Application Priority Data
Aug. 13, 2020 (CN) 202010810464.4

(51) Int. Cl. A61K 39/395

(52) U.S. Cl. CPC A61K 39/3955

(57) ABSTRACT

The present invention provides an inhibitor, an inhibitor composition, a drug and use thereof, and discloses that zkscan3 interacts with GATA1 and inhibits the transcription activity of GATA1, thereby affecting the production of red blood cells. Also, the invention also discloses that the zkscan3 gene can cause the denucleation obstacle of red blood cells by promoting the expression of KLF1. Based on this, the inhibitor of the present invention can affect the production and maturation of red blood cells by regulating the transcription of GATA1 and KLF1.

### FIELD OF THE INVENTION

The present invention relates to the technical field of biomedicines, and more particularly to an inhibitor, an inhibitor composition, a drug and use thereof.

### DESCRIPTION OF RELATED ART

ZKSCAN3 is a zinc finger protein with KRAB and SCAN domains, belonging to the Krüppel-associated box domain zinc finger protein (KRAB-ZFP) family, which is involved in a variety of cell life activities, such as cell proliferation, apoptosis and neoplastic transformation. KRAB-ZFP has four subfamilies, two with KRAB and C2H2 zinc finger motifs, and the other two with an additional SCAN (SCAN-ZFP) at the N-terminus. The KRAB domain has the function of inhibiting gene transcription, while the C2H2 zinc finger domain binds to a specific DNA sequence. The function of the SCAN domain is still poorly understood. It has been reported that ZKSCAN3 promotes the proliferation, migration and invasion of cancer cells by up-regulating the expression of genes related to cell cycle, cell proliferation, migration, angiogenesis and proteolysis.

At present, zkscna3 is still a candidate gene for cancers, and it has also been found that the Zkscan3 gene plays an important role in many important cell biology and tumor pathogenesis. At present, the research on zkscan3 gene is becoming more and more active in the world. However, under normal physiological conditions, the function of the zkscan3 gene is poorly understood.

### SUMMARY OF THE INVENTION

An objective of the present invention is to provide an inhibitor, an inhibitor composition, a drug and use thereof, wherein the inhibitor can affect the production and maturation of red blood cells by regulating the transcription of GATA1 and KLF1.

For the above purpose, the present invention utilizes the following technical solution: an inhibitor, wherein the inhibitor inhibits the expression of the Zkscan3 gene to reduce the level of an expression product of the Zkscan3 gene; or, the inhibitor binds to an expression product of the Zkscan3 gene to reduce the activity of the product of the Zkscan3 gene to promote the production and/or maturation of red blood cells.

Preferably, the inhibitor is selected from the group consisting of antibodies, functional fragments of antibodies, peptides, peptidomimetics and any combination thereof.

Preferably, the inhibitor is selected from the group consisting of gene interference, gene editing, gene silencing, gene knockout materials and any combination thereof.

Preferably, the inhibitor is selected from the group consisting of DNA, RNA, PNA, DNA-RNA-hybrids and any combination thereof.

The present invention also provides use of the inhibitor in the preparation of a drug for inhibiting the production and/or maturation of red blood cells.

The present invention also provides an inhibitor composition, including the inhibitor as an active ingredient.

Preferably, the inhibitor composition also includes a pharmaceutically acceptable excipient, which stabilizes the inhibitor and/or enhances the effect of the inhibitor.

The present invention also provides a drug, including the inhibitor, or including the inhibitor composition.

Preferably, the dosage form of the drug is selected from infusions, powders, lotions, tinctures, oils, emulsions, ointments, plasters, aerosols and any combination thereof.

The present invention also provides use of the drug in inhibiting the production and/or maturation of red blood cells.

As compared with the prior art, the invention has following beneficial effects: the present invention reveals that zkscan3 interacts with GATA1 and inhibits the transcription activity of GATA1, thereby affecting the production of red blood cells. Also, it reveals that the zkscan3 gene can cause the denucleation obstacle of red blood cells by promoting the expression of KLF1. Based on this, the inhibitor of the present invention can affect the production and maturation of red blood cells by regulating the transcription of GATA1 and KLF1.

The forgoing description is only used for summarizing the technical solution of the present invention, and in order to more clearly understand the technical means of the present invention and implement the present invention according to the content of the description, preferred examples of the present invention are described below in detail in connection with the accompanying drawings.

### BRIEF DESCRIPTION OF THE DRAWINGS

FIG. 1 is a diagram showing experimental steps for studying the effect of the zkscan3 gene on the production and/or maturation of red blood cells according to an embodiment of the present invention;

FIG. 2A is a schematic diagram of a KO mouse model according to an embodiment of the present invention.

FIGS. 2B to 2E show the flow cytometric analysis of red blood cells in spleen and bone marrow of mice according to an embodiment of the present invention;

FIG. 2F shows the detection of RBC, reticulocyte, and MCHC contents in peripheral blood of mice according to an embodiment of the present invention;

FIG. 3A is a schematic diagram of a mouse experiment with phenylhydrazine induction according to an embodiment of the present invention;

FIG. 3B is a schematic diagram of the spleens of WT and KO mice 3 days after phenylhydrazine induction according to an embodiment of the present invention;

FIG. 3C is a schematic diagram of the spleen weight/body weight percents of mice according to an embodiment of the present invention;

FIG. 3D and FIG. 3E are schematic diagrams of cell numbers of red blood cell subpopulations in spleen and bone marrow of mice according to an embodiment of the present invention;

FIGS. 3F to 3H show the detection of RBC, MCHC and reticulocyte contents in peripheral blood of mice according to an embodiment of the present invention;

FIG. 4A is a differential gene clustering map of mouse cells according to an embodiment of the present invention;

FIG. 4B is a differential gene volcano map of mouse cells according to an embodiment of the present invention;

FIG. 4C is a schematic diagram showing up-regulation and down-regulation of differential gene expression of samples according to an embodiment of the present invention;

FIG. 4D is a schematic diagram showing Q-PCR verification of red blood cells in mouse spleen according to an embodiment of the present invention;

FIGS. 5A and 5B are schematic diagrams showing fluorescence detection of GATA1, KLF1, and Tiam1 promoters according to an embodiment of the present invention;

FIGS. 5C and 5D are schematic diagrams showing western blot analysis between zkscan3 and GATA1 according to an embodiment of the present invention;

FIGS. 5E and 5F are schematic diagrams showing chromosome immunoprecipitation results between GATA1 and zkscan3 according to an embodiment of the present invention;

FIGS. 5G and 5H are schematic diagrams showing apoptosis of red blood cells in bone marrow and spleen of mice according to an embodiment of the present invention;

FIG. 5I and FIG. 5J are schematic diagrams showing CHIP analysis between zkscan3 and Tiam1 according to an embodiment of the present invention; and

FIG. 5K is a schematic diagram showing CHIP analysis between zkscan3 and KLF1 according to an embodiment of the present invention.

### DETAILED DESCRIPTION OF THE INVENTION

A detailed description of the present invention will be further given below in detail with reference to the accompanying drawings and examples. The following examples are merely used for illustrating the present invention, and not intended to limit the scope of the present invention.

The present invention has conducted a detailed study on the zkscan3 gene, and found that the zkscan3 gene plays an important regulatory role in the production and/or maturation of red blood cells. Based on this, the present invention provides an inhibitor, wherein the inhibitor can inhibit the expression of the zkscan3 gene to reduce the level of an expression product of the zkscan3 gene; or, the inhibitor can bind to an expression product of the zkscan3 gene to reduce the activity of the product of the zkscan3 gene in promoting the production and/or maturation of red blood cells.

In the present invention, the type of inhibitor that can inhibit the expression of the zkscan3 gene or can bind to the expression product of the zkscan3 gene is not particularly limited, as long as it can silence the expression of the zkscan3 gene or inhibit the function of the zkscan3 gene to promote the production and/or maturation of red blood cells. For example, the inhibitor is selected from antibodies, functional fragments of antibodies, peptides, peptidomimetics or any combination thereof; or, the inhibitor is selected from any one of gene interference, gene editing, gene silencing, or gene knockout materials; or, the inhibitor is selected from DNA, RNA, PNA, DNA-RNA-hybrids or any combination thereof.

The present invention also provides an inhibitor composition, which includes the inhibitor as an active ingredient, and also includes a pharmaceutically acceptable excipient, which stabilizes the inhibitor and/or enhances the effect of the inhibitor. Specifically, the pharmaceutically acceptable excipient is selected from biocompatible high molecular polymers, mixtures or copolymers of high molecular polymers and any combination thereof, such as a copolymer of polylactic acid, polyglycolic acid and glycolic acid, a copolymer of p-carboxyphenylpropane and sebacic acid, or ethylene vinyl acetate copolymer.

The present invention also provides a drug, including the inhibitor, or including the inhibitor composition. According to the common methods for pharmaceutical preparation, the drug is prepared into oral preparations, injections, tablets or sustained-release preparations, specifically, such as, but not limited to, suspensions, ointments, capsules, pills, tablets, or injections, etc.; and takes various shapes, such as, but not limited to, granular, flake, spherical, massive, needle, rod, and film. The above dosage forms and shapes are suitable for compositions with or without additives, and the pharmaceutical preparations are prepared by conventional preparation methods in the art. The dosage form of the drug is selected from the group consisting of infusions, powders, lotions, tinctures, oils, emulsions, ointments, plasters, aerosols and any combination thereof.

The administration dosages of the pharmaceutical preparations can be appropriately variable according to a subject to be administered, a route of administration (e.g., oral, intravenous, local injection) or a preparation form of a medicine, provided that that the pharmaceutical composition can reach an effective blood drug concentration in the mammalian body.

### Embodiment

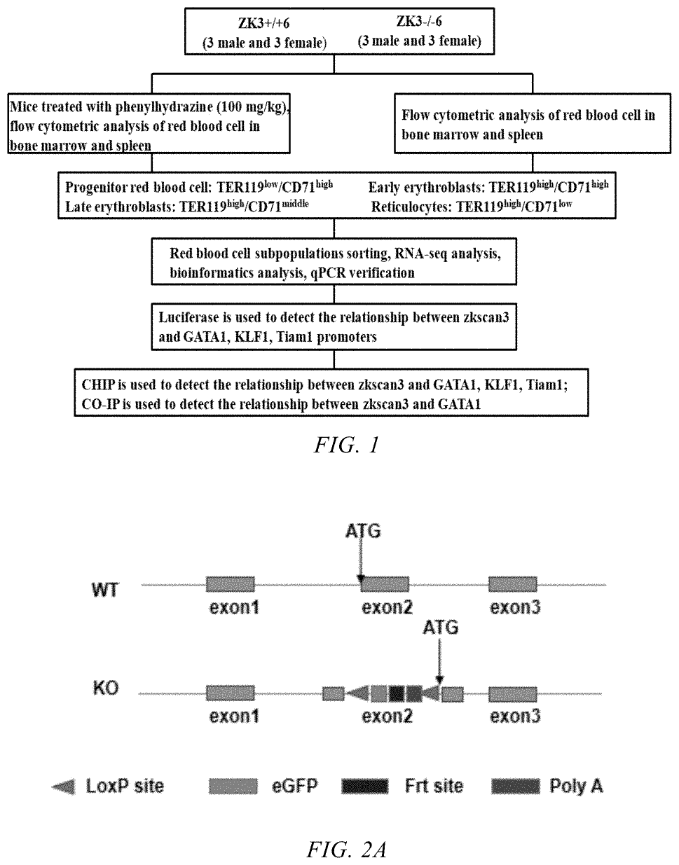

Please referring to FIG. 1, this embodiment is implemented using the following steps:

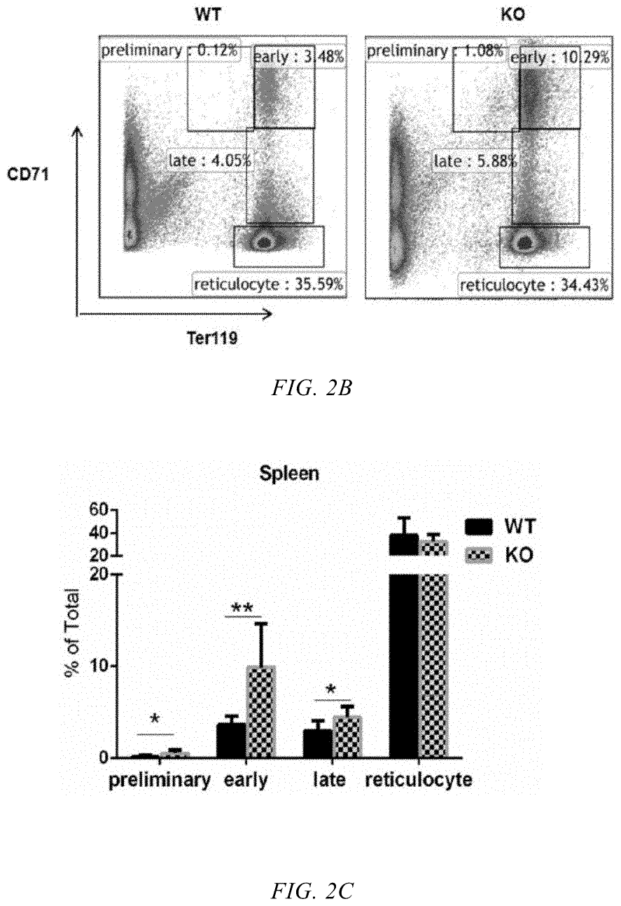

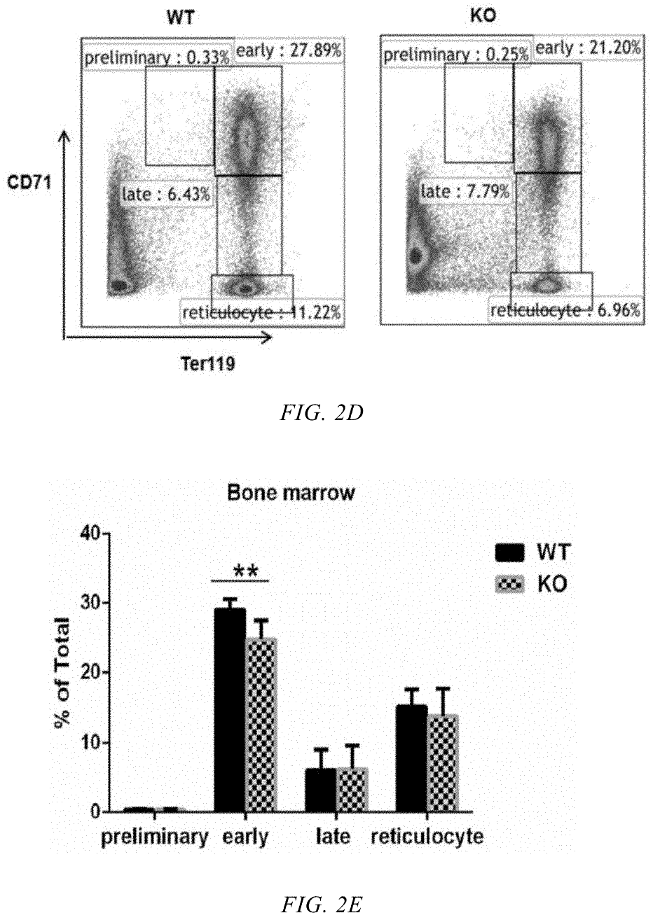

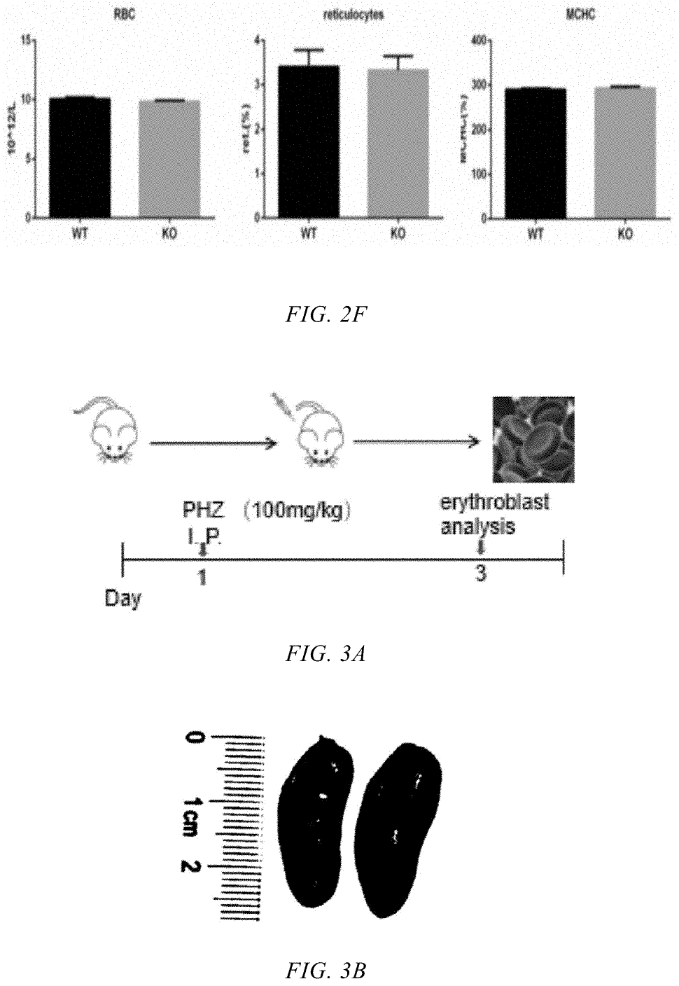

1) Flow cytometry was used to detect the percentage of red blood cell subpopulations in bone marrow and spleen of zkscan3 knockout mice and wild mice. As shown in FIGS. 2A-2F. (2B-2E) show flow cytometric analysis of the percentage of red blood cells in spleen and bone marrow of mice with statistical analysis (n=10). (2F) shows the detection of RBC, reticulocytes and MCHC in peripheral blood of mice by a five-classification hematology analyzer. The results show that the percentages of early and late erythroblasts in the spleen of zkscan3 knockout mice are increased. The percentages of early erythroblasts in the bone marrow are reduced. There is no significant difference in peripheral blood for other subpopulations of red blood cells.

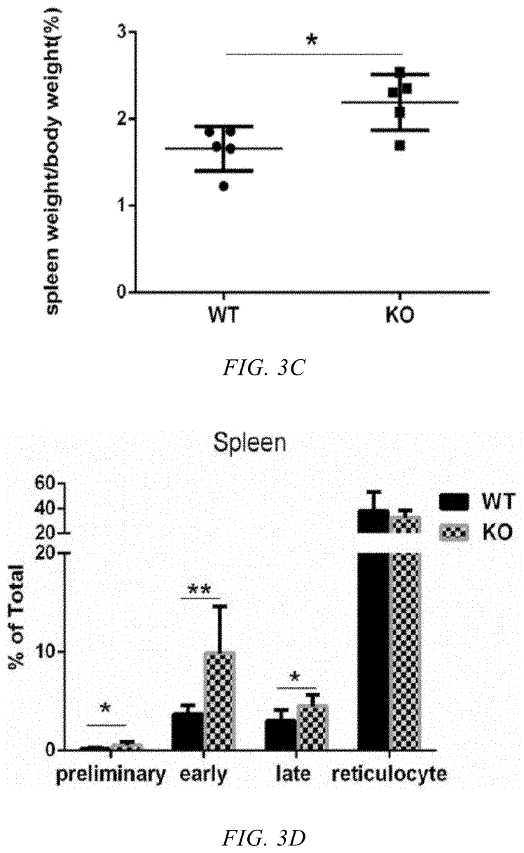

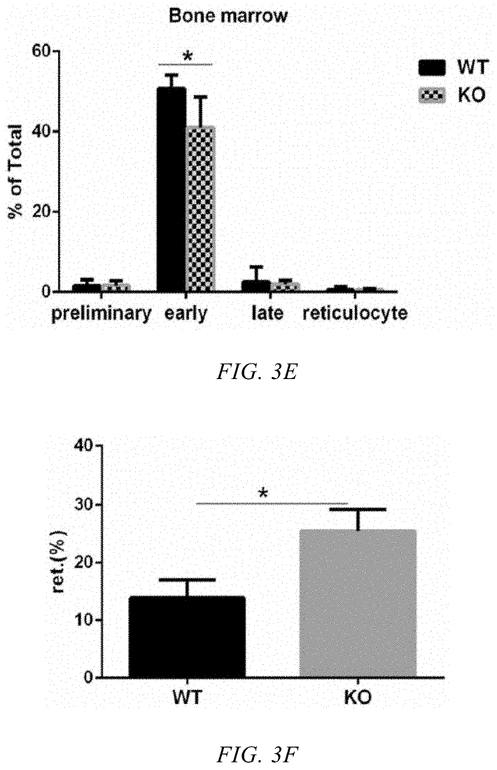

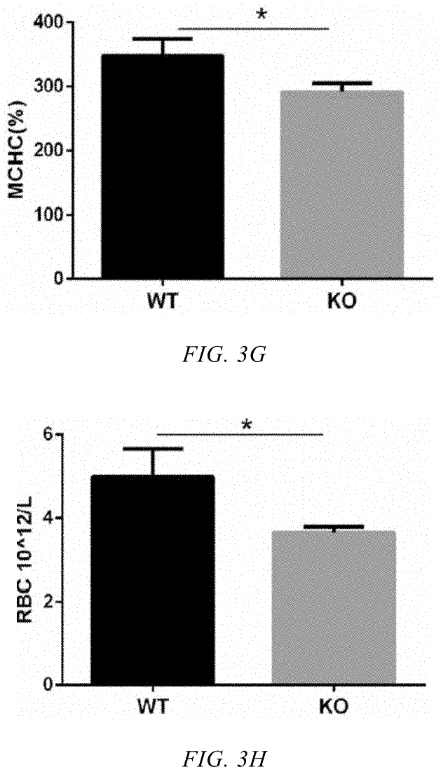

2) Flow cytometry was used to detect red blood cells in spleen and bone marrow, and a five-classification hematology analyzer was used to detect those in peripheral blood of mice. Specifically, 6 WT and KO mice aged 6-8 weeks were selected, and PHZ (100 mg/kg) (n=6) was intraperitoneally injected. 3 days later, red blood cells in the bone marrow, spleen and peripheral blood of the mice were detected by flow cytometry. As shown in FIG. 3A-FIG, 3H. (3A) shows a schematic diagram of a mouse experiment with phenylhydrazine induction. (3B) shows spleens of WT and KO mice after 3 days. (3C) shows spleen weight/body weight percent of mice. (3D-3E) show cell numbers of red blood cell subpopulations in the spleen and bone marrow. (3F-3H) show RBCs, MCHCs, and reticulocytes in peripheral blood. Compared with wild-type mice, zkscan3 knockout mice have larger spleens, the percentages of early and late erythroblasts in the spleen are significantly increased, the percentages of early erythroblasts in the bone marrow are decreased, and the reticulocytes in the peripheral blood (%) are significantly increased, MCHC (%) is significantly decreased, and the number of RBCs is decreased.

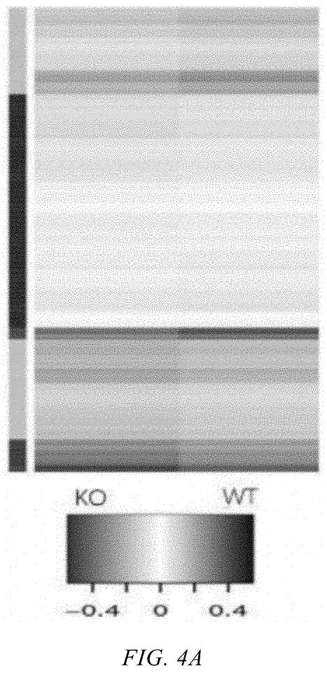

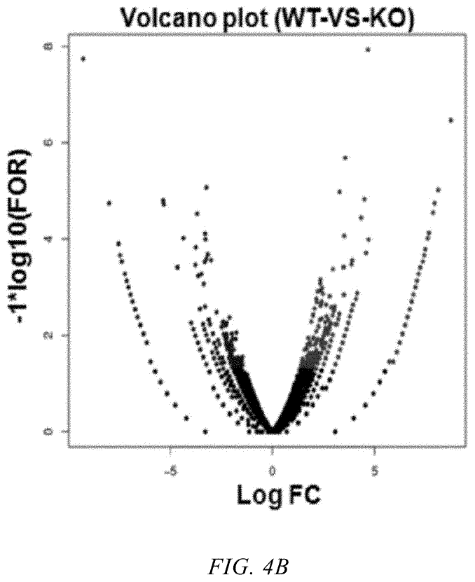

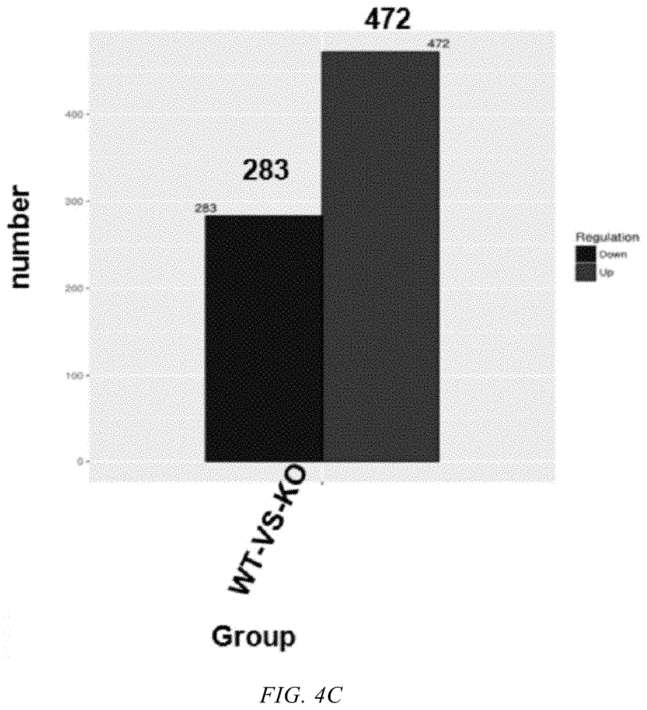

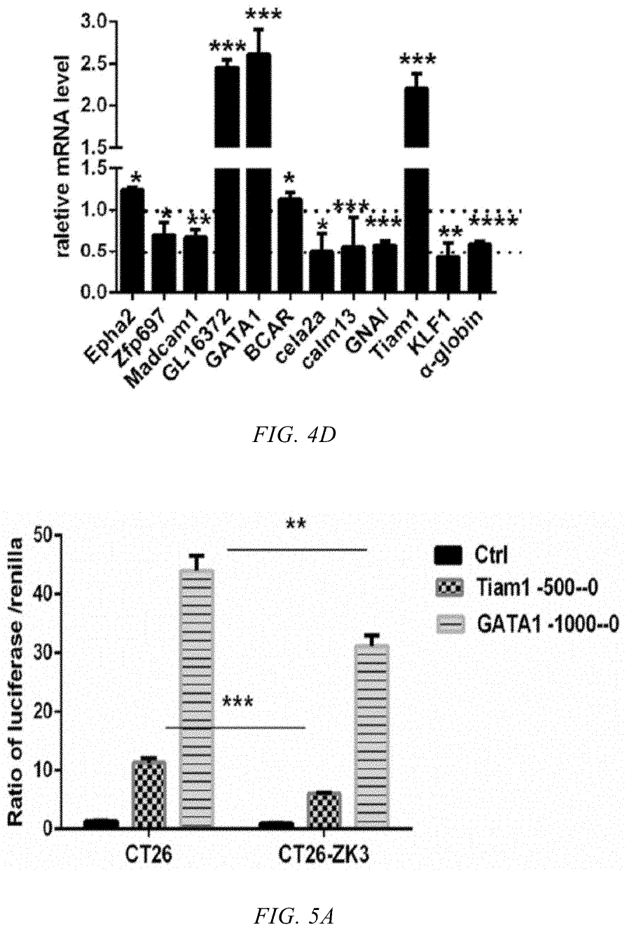

3) Referring to FIGS. 4A-4D, red blood cells in the spleen were sorted and RNA-seq analysis was performed. There are 283 genes down-regulated and 472 genes up-regulated in the red blood cells of zkscan3 knockout mice. Then, the differential expression of key genes was verified by qPCR. The expressions of madcaml, zfp697, cela2a, calm13, GNAI, KLF1 and a-globin are decreased. The expression levels of Epha2, GL16372, GATA1, Tiam1 and BCAR are increased. Among them, (4A) shows a differential gene clustering map, where Log10 (RPKM+1) value is used for clustering, blue indicates high-expressed genes, and red indicates low-expressed genes. The color changes from red to blue, indicating higher gene expression. (4B) shows a differential gene volcano map, where the red dots of significantly different genes indicate up-regulation, and blue dots indicate down-regulation. The abscissa represents the fold change of gene expression in different samples. The ordinate represents the statistical significance of the difference in gene expression. (4C) shows up-regulation and down-regulation of differential gene expression of samples. (4D) shows Q-PCR verification of gene expression in red blood cells of the spleen.

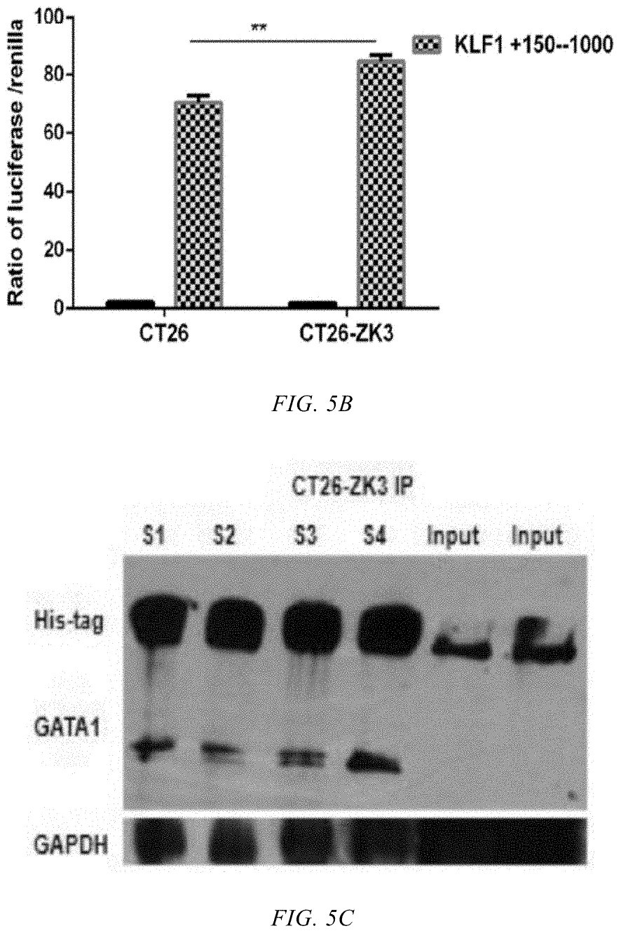

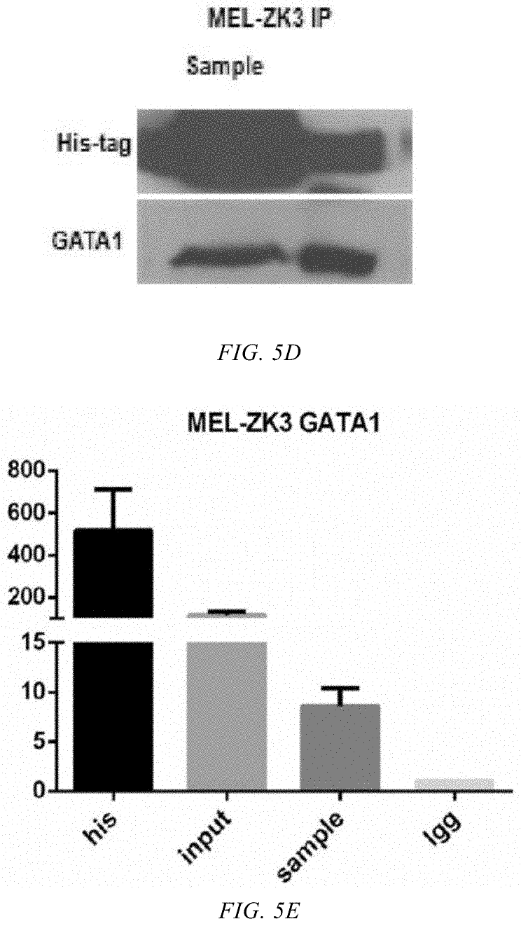

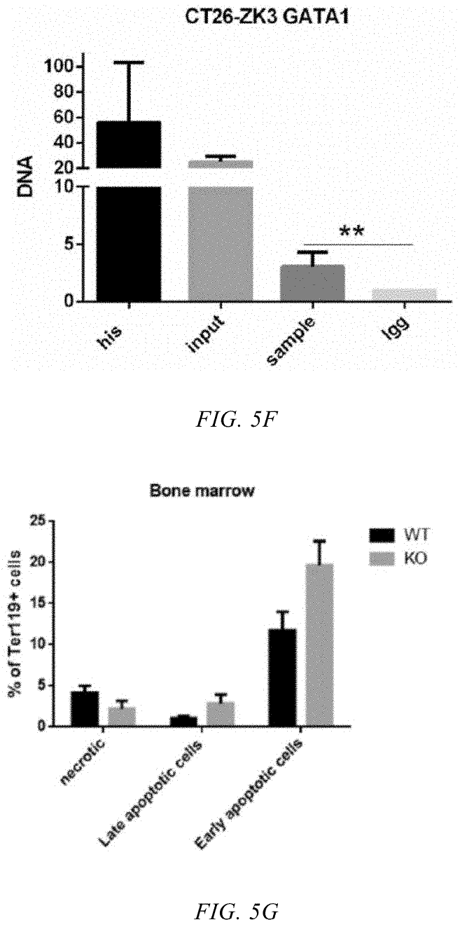

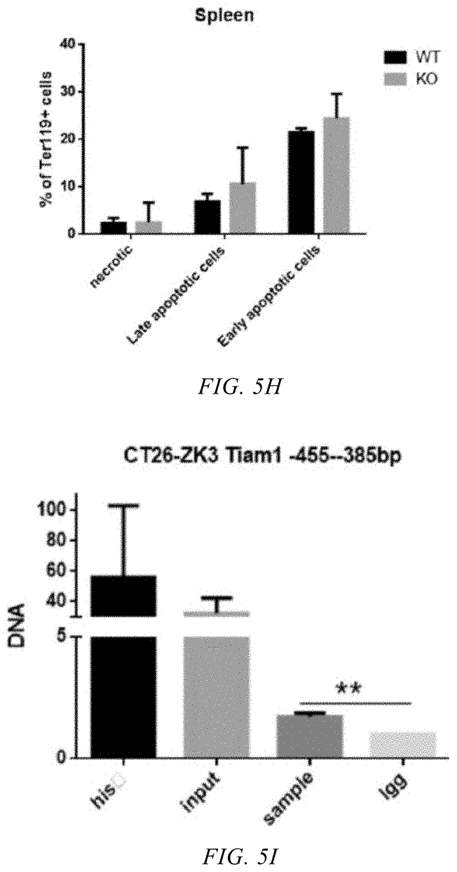

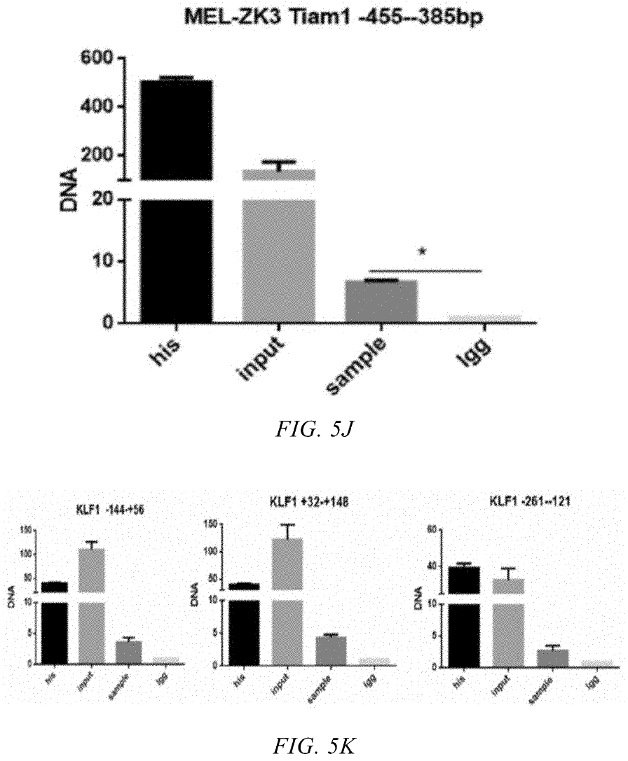

4) Given that GATA1 and KLF1 are indispensable in the development of red blood cells. Therefore, the relationship between zkscan3 and GATA1, KLF1 was studied. First, luciferase was used to detect the relationship between zkscan3 and GATA1, KLF1 promoters. The luciferase signal of GATA1 is increased, and the luciferase signal of KLF1 is decreased. The relationship between zkscan3 and GATA1, KLF1 was further verified by CHIP and CO-IP. Zkscan3 interacts with GATA1 and KLF to promote the transcriptional activity of GATA1 and inhibit the activity of KLF1. Referring to FIG. 5, (5A-5B) show the targeting effect of zkscan3 on GATA1, KLF1, and Tiam1 promoters, studied by luciferase reporter gene experimental analysis (**P<0.01). (5C-5D) show western blot analysis of the relationship between zkscan3 and GATA1 after protein extraction of CT26-ZK3 cells and MEL-ZK3 cells and then immunoprecipitation. (5E-5F) show chromosome immunoprecipitation (ChIP) confirmation of the relationship between GATA1 and zkscan3. (5G-5H) show apoptosis of red blood cells in the bone marrow and spleen of mice. (5I-5J) show CHIP analysis of the interaction between zkscan3 and Tiaml in CT26-zk3 and MEL-zk3 cells. (5K) shows CHIP detection of the relationship between zkscan3 and KLF1 in MEL-ZK3 cells.

The technical features of the above-mentioned examples can be combined arbitrarily. In order to make the description concise, all possible combinations of the various technical features in the above examples are not described. However, as long as there is no contradiction in a combination of these technical features, it should be considered that the combination is within the scope of this description.

The examples described above, representing only a few implementations of the present invention, are specifically described in detail above, but they are not to be construed as limiting the scope of the present invention. It should be noted that several variations and modifications can be made by those of ordinary skill in the art without departing from the concept of the present invention, and that these variations and modifications all fall within the scope of protection of the present invention. Therefore, the protection scope of the present invention is subject to the protection scope of the appended claims.

1. An inhibitor, wherein the inhibitor inhibits the expression of the Zkscan3 gene to reduce the level of an expression product of the Zkscan3 gene; or, the inhibitor binds to an expression product of the Zkscan3 gene to reduce the activity of the product of the Zkscan3 gene to promote the production and/or maturation of red blood cells.

2. The inhibitor of claim 1, wherein the inhibitor is selected from the group consisting of antibodies, functional fragments of antibodies, peptides, peptidomimetics and any combination thereof.

3. The inhibitor of claim 1, wherein the inhibitor is selected from the group consisting of gene interference, gene editing, gene silencing, gene knockout materials and any combination thereof.

4. The inhibitor of claim 1, wherein the inhibitor is selected from the group consisting of DNA, RNA, PNA, DNA-RNA-hybrids and any combination thereof

5. Use of the inhibitor of claim 1 in the preparation of a drug for inhibiting the production and/or maturation of red blood cells.

6. An inhibitor composition, comprising the inhibitor of claim 1 as an active ingredient.

7. The inhibitor composition of claim 6, where the inhibitor composition comprises a pharmaceutically acceptable excipient, which stabilizes the inhibitor and/or enhances the effect of the inhibitor.

8. A drug, comprising the inhibitor of claim 1.

9. The drug of claim 8, wherein the dosage form of the drug is selected from infusions, powders, lotions, tinctures, oils, emulsions, ointments, plasters, aerosols and any combination thereof.

10. Use of the drug of claim 9 in inhibiting the production and/or maturation of red blood cells.
